(19) United States
(12) Patent Application Publication
(10) Pub. No.: US 2004/0161395 A1
(43) Pub. Date: Aug. 19, 2004

(54) Cosmetic compositions containing composite siloxane polymers

(76) Inventors: Anjali Abhimanyu Patil, Westfield, NJ (US); Joseph Frank Calello, Bridgewater, NJ (US); Robert Walter Sandewicz, Monroe Township, NJ (US); Jean Marie Manelski, Spring Lake, NJ (US); Kaya Teray McIntosh, Willingboro, NJ (US)

Correspondence Address: Julie Blackburn, Revlon Consumer Products Corporation, 237 Park Avenue, New York, NY, 10017, US

(21) Appl. No.: 10/367,301

(22) Filed: Feb. 14, 2003

(51) Int. Cl. A61K 7/025; A61K 7/06; A61K 7/11

(52) U.S. Cl. 424/70.12; 424/64

(57) ABSTRACT

A cosmetic composition for application to keratinous surfaces for the purpose of coloring, conditioning, or protecting such keratinous surface, comprising a silicone polymer which is the reaction product of a siloxane resin and an diorganosiloxane, in a cosmetically acceptable carrier and method for treating such the keratinous surface for improvement.

### TECHNICAL FIELD

The invention is in the field of cosmetic compositions containing certain composite siloxane polymers.

### BACKGROUND OF THE INVENTION

Film forming compositions are widely used in cosmetics. There are many reasons for desiring to form a film on keratinous surfaces. For example, it may be desirable to form a color film on lips with lipsticks or lipglosses; or a film on facial skin with foundation makeups, lotions, or creams; or on nails with nail enamel or nail conditioners. Accordingly, the nature and integrity of the film formed on the keratinous surface is critical depending on the end benefit that is to be delivered by the cosmetic composition applied to the keratinous surface. Cosmetics companies are continually interested in improving the function and integrity of cosmetic films formed on the skin.

In most cases, the reason for forming the film is to provide some aesthetic or functional end result such as retention of moisture on the underlying keratinous surface, or to improve the appearance of skin imperfections, lines, wrinkles, sags, discolorations, and the like; or to improve aesthetics such as gloss, shine, wear, or transfer resistance in cosmetics such as lipsticks or foundations.

Usually the integrity and functionality of the cosmetic film formed depends on the type and amount of polymers in the cosmetic composition. Some polymers are better at providing certain effects than others, and cosmetics companies will often select and even tailor polymers to achieve the desired objectives in the cosmetics they are formulating.

Still, there is a need for cosmetic compositions that exhibit improved properties on the skin in terms of improving the moisture barrier properties of skin, or improving the appearance of skin imperfections, lines, wrinkles, discolorations, and the like; or improving aesthetics such as gloss and transfer resistance of films formed.

It is objective of the invention to provide cosmetic compositions that provide improved films on skin.

It is another object of the invention to provide cosmetic compositions that provide films that improve the superficial appearance of skin or lips, for example, by improving the appearance of skin imperfections, lines, wrinkles, sags, discolorations, and the like on keratinous surfaces.

It is another object of the invention to provide cosmetic compositions for improving the aesthetics of cosmetic films formed on keratinous surfaces, such aesthetics including gloss, shine, transfer resistance, and wear.

It is an object to provide a cosmetic compositions containing a certain type of polymer that provides these desired benefits.

### SUMMARY OF THE INVENTION

A cosmetic composition for application to keratinous surfaces for the purpose of coloring, conditioning, or protecting such keratinous surface, comprising a silicone copolymer which is the reaction product of a siloxane resin and an diorganosiloxane, in a cosmetically acceptable carrier.

The invention is also directed to a method for improving the properties of cosmetic films applied to keratinous surfaces comprising including in said cosmetic composition at least one silicone copolymer which is the reaction product of a siloxane resin and a diorganosiloxane.

### DETAILED DESCRIPTION

The invention is directed to a cosmetic composition for application to keratinous surfaces to color, condition, or protect the surfaces, comprising a silicone copolymer which is the reaction product of a siloxane resin and an diorganosiloxane in a cosmetically acceptable carrier.

The term “reaction product” when used herein is used in the broad sense and refers to any reaction between the siloxane resin and a diorganosiloxane that will yield the siloxane copolymer used in the claimed method and compositions of the invention.

The term “diorganosiloxane” means a linear, or branched, cross-linked or uncrosslinked, organosiloxane polymer containing at least monofunctional (“M”) and/or difunctional (“D”) units, e.g. dimethylsiloxane, dimethyl-methylhydrogen siloxane, etc.

The term “siloxane resin” means an organosiloxane containing at least trifunctional (“T”) units or quadrifunctional (“Q”) units, either alone or in combination with monofunctional units.

The term “moisture barrier properties” means that ability of the skin to retain moisture, in other words the ability of the skin to exhibit reduced transepidermal water loss. More specifically, application of the cosmetic composition of the invention will improve the moisture barrier properties of the skin when compared to untreated skin.

The term “gloss” or “shine” means the shininess of the cosmetic layer.

The term “transfer resistant” refers to the long wearing characteristics of the film, or the ability of the film to resist transfer when the keratinous surface containing the cosmetic film thereon is touched to another surface.

The term “wear” means the length of time that the cosmetic composition remains on the skin during normal activities engaged in by the wearer of the cosmetic.

The term “spreadability” means the ease with which the cosmetic composition spreads on the skin when it is applied. It is important that cosmetics spread readily on the surfaces to which they are applied, but not run onto undesired areas. When incorporated into the claimed compositions or used in the claimed method, the composite silicone copolymer, in some cases, provides improved spreadability on the skin.

The term “keratinous surfaces” when used herein means any bodily surface covered by keratinous cells, including skin, nails, or hair.

The term “skin” when used herein means facial and body skin surfaces as well as lips.

The term “volatile” means that an ingredient has a vapor pressure of at least about 2 mm. of mercury at 20° C.

The term “non-volatile” means that an ingredient has a vapor pressure of less than about 2 mm. of mercury at 20° C.

All percentages set forth herein are percentages by weight unless otherwise indicated.

The cosmetics and method will be further described herein.

I. The Cosmetic Compositions

The claimed cosmetic composition comprises a silicone copolymer obtained by condensation of a siloxane resin and a diorganosiloxane in a cosmetically acceptable carrier.

A. The Silicone Copolymer

The silicone copolymer used in the method and compositions of the invention comprises the reaction product of a siloxane resin and a diorganosiloxane. Preferably, the amount of silicone copolymer in the compositions ranges from about 0.001 to 75%, preferably about 0.01-60%, more preferably about 0.1-50% by weight of the total composition.

Preferably, the siloxane resin is comprised of T or Q units, which may have M units and D units; and the diorganosiloxane is comprised of M and D units.

The term “M unit” means a monofunctional unit, which is a siloxy unit that contains one silicon atom bonded to one oxygen atom, with the remaining three substituents on the silicon atom being other than oxygen. In particular, in a monofunctional siloxy unit, the oxygen atom present is shared by 2 silicon atoms when the monofunctional unit is polymerized with one or more of the other units. In silicone nomenclature used by those skilled in the art, a monofunctional siloxy unit is designated by the letter “M”, and means a unit having the general formula:

R₁R₂R₃SiO_{1/2}

wherein R₁, R₂, and R₃ are each independently C₁₋₃₀, preferably C₁₋₁₀, more preferably C₁₋₄ straight or branched chain alkyl, or C₁₋₃₀, preferably C₁₋₁₀, more preferably C₁₋₄ alkoxy, which may be substituted with phenyl or one or more hydroxyl groups; phenyl; carboxylic esters; or hydrogen. The SiO_{1/2} designation means that the oxygen atom in the monofunctional unit is bonded to, or shared, with another silicon atom when the monofunctional unit is polymerized with one or more of the other types of units. For example, when R₁, R₂, and R₃ are methyl the resulting monofunctional unit is of the formula:

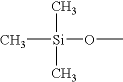

When this monofunctional unit is polymerized with one or more of the other units the oxygen atom will be shared by another silicon atom, i.e. the silicon atom in the monofunctional unit is bonded to ½ of this oxygen atom.

The term “difunctional siloxy unit” is generally designated by the letter “D” in standard silicone nomenclature. If the D unit is substituted with substituents other than methyl the “D” designation is sometimes used, which indicates a substituent other than methyl. For purposes of this disclosure, a “D” unit has the general formula:

R₁R₂SiO_{2/2}

wherein R₁ and R₂ are defined as above. The SiO_{2/2} designation means that the silicon atom in the difunctional unit is bonded to two oxygen atoms when the unit is polymerized with one or more of the other units. For example, when R₁, R₂, are methyl the resulting difunctional unit is of the formula:

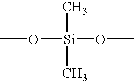

When this difunctional unit is polymerized with one or more of the other units the silicon atom will be bonded to two oxygen atoms, i.e. will share two one-halves of an oxygen atom.

The term “trifunctional siloxy unit” is generally designated by the letter “T” in standard silicone nomenclature. A “T” unit has the general formula:

R₁SiO_{3/2}

wherein R₁ is as defined above. The SiO_{3/2} designation means that the silicon atom is bonded to three oxygen atoms when the unit is copolymerized with one or more of the other units. For example when R₁ is methyl the resulting trifunctional unit is of the formula:

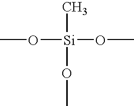

When this trifunctional unit is polymerized with one or more of the other units, the silicon atom shares three oxygen atoms with other silicon atoms, i.e. will share three halves of an oxygen atom.

The term “tetrafunctional siloxy unit” is generally designated by the letter “Q” in standard silicone nomenclature. A “Q” unit has the general formula:

SiO_{4/2}

The SiO_{4/2} designation means that the silicon shares four oxygen atoms (i.e. four halves) with other silicon atoms when the tetrafunctional unit is polymerized with one or more of the other units. The SiO_{4/2} unit is best depicted as follows:

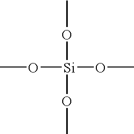

The silicone polymer used in the composition are made according to processes well known in the art. In general siloxane polymers are obtained by hydrolysis of silane monomers, preferably chlorosilanes. The chlorosilanes are hydrolyzed to silanols and then condensed to form siloxanes. For example, Q units are often made by hydrolyzing tetrachlorosilanes in aqueous or aqueous/alcoholic media to form the following:

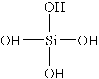

The above hydroxy substituted silane is then condensed or polymerized with other types of silanol substituted units, in this units including diorganosiloxane units, such as:

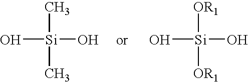

Wherein R₁ is as defined above.

Because the hydrolysis and condensation may take place in aqueous or aqueous/alcoholic media wherein the alcohols are preferably lower alkanols such as ethanol, propanol, or isopropanol, the units may have residual hydroxyl or alkoxy functionality. Preferably, the polymers are made by hydrolysis and condensation in aqueous/alcoholic media, which provides resins that have residual silanol and alkoxy functionality. In the case where the alcohol is ethanol, the result is a resin that has residual hydroxy or ethoxy functionality on the siloxane polymer. The silicone film forming polymers used in the compositions of the invention are generally made in accordance with the methods set forth in Silicon Compounds (Silicones), Bruce B. Hardman, Arnold Torkelson, General Electric Company, Kirk-Othmer Encyclopedia of Chemical Technology, Volume 20, Third Edition, pages 922-962, 1982, which is hereby incorporated by reference in its entirety.

The composite polymer used in the compositions of the invention may also be made as follows:

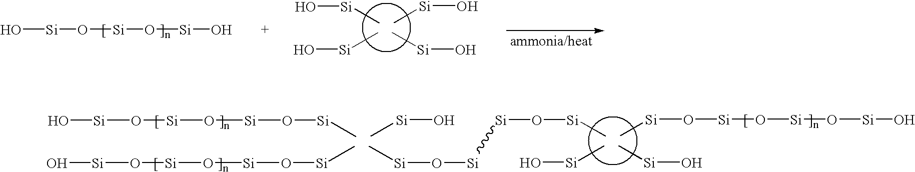

If desired, the hydroxy functional groups on the molecule may be further reacted to form alkoxy groups, alkyl groups, halogens, which may be substituted with one or more substituents such as hydroxyl, and so on.

Most preferred is where the siloxane copolymer is obtained by reacting a diorganosiloxane having terminal hydroxyl groups with a siloxane resin having hydroxyl groups by combining the reactants in the presence of heat and ammonia, as set forth in U.S. Pat. No. 4,584,355, which is herby incorporated by reference in its entirety.

Particularly preferred are silicone copolymers manufactured by Dow Corning which are sold under the tradename BIO-PSA, series 4100, 4200, 4300, 4400, 4500 or 4600. Most preferred are the Dow Corning silicone copolymers sold under the BIO-PSA 4500 Series which are standard medium tack silicone copolymers. Most preferred is BIO-PSA 4505. Certain of such polymers are described by CAS numbers 68440-70-0 (trimethylated silica treated with dimethyl siloxane), 238094-36-5 (methylated trimethylated silica).

B. The Cosmetically Acceptable Carrier.

A wide variety of cosmetically acceptable carriers are suitable depending on the end use of the cosmetic composition. The carrier may include hair compositions such as shampoos, hair conditioners, hair sprays, hair mousses and gels, or hair color. The carrier may include skin and body lotions, creams, gels, or sprays; or sunscreen compositions in the same form. The carrier may also include a wide variety of colored cosmetic products such as lipstick, blush, eyeshadow, foundation makeup, concealer, mascara, and the like. Also suitable for the cosmetically acceptable carrier are nail compositions such as nail enamel, nail treatment products, cuticle treatment products, and the like. In the most preferred embodiment, the cosmetically acceptable carrier is free of silicone elastomers.

1. Compositions Applied to Hair

Suitable cosmetically acceptable carriers include those which are applied to hair such as shampoos, conditioners, hair sprays, hair color, and the like.

(a) Shampoos

Typically, shampoos are in the aqueous form and comprise about 0.1-99.9% water and about 0.01-50%, preferably about 0.1-40%, more preferably about 0.5-35% of a cleansing surfactant. Suitable cleansing surfactants include anionic, amphoteric, zwitterionic, or nonionic surfactants that are capable of providing a certain cleansing or lathering function.

(i) Surfactants

(aa) Anionic Surfactants

Anionic surfactants include alkyl and alkyl ether sulfates generally having the formula ROSO₃M and RO(C₂H₄o)ₓSO₃M wherein R is alkyl or alkenyl of from about 10 to 20 carbon atoms, x is 1 to about 10 and M is a water soluble cation such as ammonium, sodium, potassium, or triethanolamine cation.

Another type of anionic surfactant which may be used in the compositions of the invention are water soluble salts of organic, sulfuric acid reaction products of the general formula:

R₁—SO₃-M

wherein R₁ is chosen from the group consisting of a straight or branched chain, saturated aliphatic hydrocarbon radical having from about 8 to about 24 carbon atoms, preferably 12 to about 18 carbon atoms; and M is a cation. Examples of such anionic surfactants are salts of organic sulfuric acid reaction products of hydrocarbons such as n-paraffins having 8 to 24 carbon atoms, and a sulfonating agent, such as sulfur trioxide.

Also suitable as anionic surfactants are reaction products of fatty acids esterified with isethionic acid and neutralized with sodium hydroxide. The fatty acids may be derived from coconut oil, for example.

In addition, succinates and succinimates are suitable anionic surfactants. This class includes compounds such as disodium N-octadecylsulfosuccinate; tetrasodium N-(1,2-dicarboxyethyl)-N-octadecylsulfosuccinate; and esters of sodium sulfosuccinic acid e.g. the dihexyl ester of sodium sulfosuccinic acid, the dioctyl ester of sodium sulfosuccinic acid, and the like.

Other suitable anionic surfactants include olefin sulfonates having about 12 to 24 carbon atoms. The term “olefin sulfonate” means a compound that can be produced by sulfonation of an alpha olefin by means of uncomplexed sulfur trioxide, followed by neutralization of the acid reaction mixture in conditions such that any sulfones which have been formed in the reaction are hydrolyzed to give the corresponding hydroxyalkanesulfonates. The alpha-olefin from which the olefin sulfonate is derived is a mono-olefin having about 12 to 24 carbon atoms, preferably about 14 to 16 carbon atoms.

Other classes of suitable anionic organic surfactants are the beta-alkoxy alkane sulfonates or water soluble soaps thereof such as the salts of C₁₀₋₂₀ fatty acids, for example coconut and tallow based soaps. Preferred salts are ammonium, potassium, and sodium salts.

Still another class of anionic surfactants include N-acyl amino acid surfactants and salts thereof (alkali, alkaline earth, and ammonium salts) having the formula:

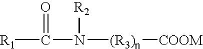

wherein R₁ is a C₈₋₂₄ alkyl or alkenyl radical, preferably C₁₀₋₁₈; R₂ is H, C₁₋₄ alkyl, phenyl, or —CH₂COOM; R₃ is CX₂— or C₁₋₂ alkoxy, wherein each X independently is H or a C₁₋₆ alkyl or alkylester, n is from 1 to 4, and M is H or a salt forming cation as described above. Examples of such surfactants are the N-acyl sarcosinates, including lauroyl sarcosinate, myristoyl sarcosinate, cocoyl sarcosinate, and oleoyl sarcosinate, preferably in sodium or potassium forms.

(bb) Nonionic Surfactants

The composition may contain one or more nonionic surfactants. Nonionic surfactants are generally compounds produced by the condensation of alkylene oxide groups with a hydrophobic compound. Classes of nonionic surfactants include:

Long chain dialkyl sulfoxides containing one short chain alkyl or hydroxy alkyl radical of from about 1 to 3 carbon atoms and one long hydrophobic chain which may be an alkyl, alkenyl, hydroxyalkyl, or ketoalkyl radical containing from about 8 to 20 carbon atoms, from 0 to 10 ethylene oxide moieties, and 0 or 1 glyceryl moiety.

Polysorbates, such as sucrose esters of fatty acids. Examples of such materials include sucrose cocoate, sucrose behenate, and so on.

Polyethylene oxide condensates of alkyl phenols, for example the condensation products of alkyl phenols having an alkyl group of 6 to 20 carbon atoms with ethylene oxide being present in amounts of about 10 to 60 moles of ethylene oxide per mole of alkyl phenol.

Condensation products of ethylene oxide with the reaction product of propylene oxide and ethylene diamine.

Condensation products of aliphatic alcohols having 8 to 18 carbon atoms with ethylene oxide, for example a coconut alcohol/ethylene oxide condensate having 10 to 30 moles of ethylene oxide per mole of coconut alcohol, the coconut alcohol fraction having 10 to 14 carbon atoms.

Long chain tertiary amine oxides such as those corresponding to the general formula:

R₁R₂R₃NO

wherein R₁ contains an alkyl, alkenyl or monohydroxyalkyl radical ranging from about 8 to 18 carbon atoms in length, from 0 to about 10 ethylene oxide moieties, and from 0 to about 1 glyceryl moiety and R₂ and R₃ are each alkyl or monohydroxyalkyl groups containing from about 1 to about 3 carbon atoms.

Long chain tertiary phosphine oxides corresponding to the general formula:

RR₁R₂PO

wherein R contains an alkyl, alkenyl, or monohydroxyalkyl radical having 8 to 18 carbon atoms, from 0-10 ethylene oxide moieties and 0 or 1 glyceryl moiety, and R₂ and R₃ are each alkyl or monohydroxyalkyl group containing from about 1 to 3 carbon atoms.

Alkyl polysaccharides having a hydrophobic group of 6 to 30, preferably 10, carbon atoms and a polysaccharide group such as glucose, galactose, etc. Suitable alkyl polysaccharides are octyl, nonydecyl, undecyldodecyl, tridecyl, tetradecyl, pentadecyl, hexadecyl, heptadecyl, and octadecyl, di-, tri-, tetra-, penta-, and hexaglucosides, galactosides, lactosides, glucoses, fructosides, fructoses, and so on.

Polyethylene glycol (PEG) glyceryl fatty esters, having the formula

RC(O)OCH₂CH(OH)CH₂(OCH₂CH₂)ₙOH

wherein n is 5-200 and RC(O)— is a hydrocarbylcarbonyl group wherein R is preferably an aliphatic radical having 7 to 19 carbon atoms.

Other nonionic surfactants that may be used include C₁₀₋₁₈ alkyl(C₁₋₆)polyhydroxy fatty acid amides such as C₁₂₋₁₈ methylglucamides, N-alkoxy polyhydroxy fatty acid amides, N-propyl through N-hexyl C₁₂₋₁₈ glucamides and so on.

(cc) Amphoteric Surfactants

Amphoteric surfactants may also be used in the compositions. They are generally described as derivatives of aliphatic secondary or tertiary amines wherein one aliphatic radical is a straight or branched chain alkyl of 8 to 18 carbon atoms and the other aliphatic radical contains an anionic group such as carboxy, sulfonate, sulfate, phosphate, or phosphonate.

Suitable amphoteric surfactants may be imidazolinium compounds having the general formula:

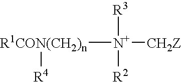

wherein R¹ is C₈₋₂₂ alkyl or alkenyl, preferably C₁₂₋₁₆; R² is hydrogen or CH₂CO₂M; R³ is CH₂CH₂OH or CH₂CH₂OCH₂CHCOOM; R⁴ is hydrogen, CH₂CH₂OH, or CH₂CH₂OCH₂CH₂COOM, Z is CO₂M or CH₂CO₂M, n is 2 or 3, preferably 2, M is hydrogen or a cation such as an alkali metal, alkaline earth metal, ammonium, or alkanol ammonium. cation. Examples of such materials are marketed under the tradename MIRANOL, by Miranol, Inc.

Also suitable amphoteric surfactants are monocarboxylates or dicarboxylates such as cocamphocarboxypropionate, cocoamphocarboxypropionic acid, cocamphocarboxyglycinate, and cocoamphoacetate.

Other types of amphoteric surfactants include aminoalkanoates of the formula

R—NH(CH₂)ₙCOOM

or iminodialkanoates of the formula:

R—N[(CH₂)ₘCOOM]₂

and mixtures thereof; wherein n and m are 1 to 4, R is C₈₋₂₂ alkyl or alkenyl, and M is hydrogen, alkali metal, alkaline earth metal, ammonium or alkanolammonium. Examples of such amphoteric surfactants include n-alkylaminopropionates and n-alkyliminodipropionates, which are sold under the trade name MIRATAINE by Miranol, Inc. or DERIPHAT by Henkel, for example N-lauryl-beta-amino propionic acid, N-lauryl-beta-iminio-dipropionic acid, or mixtures thereof.

(dd) Zwitterionic Surfactants

Zwitterionic surfactants are also suitable for use in the shampoo compositions of the invention. The general formula for such surfactants is:

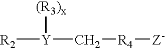

wherein R₂ contains an alkyl, alkenyl or hydroxy alkyl radical of from about 8 to about 18 carbon atoms, from 0 to about 10 ethylene oxide moieties and 0 or 1 glyceryl moiety; Y is selected from the group consisting of nitrogen, phosphorus, and sulfur atoms; R₃ is an alkyl or monohydroxyalkyl group containing about 1 to 3 carbon atoms; X is 1 when Y is a sulfur atom, and 2 when Y is a nitrogen or phosphorus atom; R₄ is an alkylene or hydroxyalkylene of from about 1 to about 4 carbon atoms, and Z is a radical selected from the group consisting of carboxylate, sulfonate, sulfate, phosphonate, and phosphate groups.

Zwitterionic surfactants include betaines, for example higher alkyl betaines such as coco dimethyl carboxymethyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxylethyl betaine, and mixtures thereof. Also suitable are sulfo- and amido-betaines such as coco dimethyl sulfopropyl betaine, stearyl dimethyl sulfopropyl betaine, and the like.

(ii) Other Ingredients

Shampoo compositions may comprise a variety of other ingredients that enhance the beneficial and aesthetic properties thereof, including oily conditioning agents, humectants, biological ingredients, other film formers, preservatives, and mixtures of such materials.

(b) Hair Conditioners

Also suitable as the cosmetically acceptable carrier is a hair conditioner. Generally hair conditioners comprise an aqueous base, one or more cationic conditioning agents, and, optionally, oily or waxy conditioning agents such as silicone oils, fatty acids, fatty alcohols and the like.

(i) Cationic Conditioning Agents

Suitable cationic conditioning agents include cationic surfactants and/or polymers. Suggested ranges of such ingredients, if present, are about 0.01-15%, preferably about 0.05-10%, more preferably about 0.10-8%. Suitable cationic conditioning agents include cationic polymers, quaternary ammonium salts, or the salts of fatty amines. Quaternary ammonium salts have the formula:

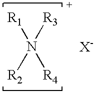

wherein R₁ is hydrogen, an aliphatic group of 1 to 22 carbon atoms, or aromatic, aryl, or alkaryl group having 12 to 22 carbon atoms; R₂ is an aliphatic group having 1-22 carbon atoms; R₃ and R₄ are each alkyl groups of from 1 to 3 carbon atoms, and X is an anion selected from halogen, acetate, phosphate, nitrate and methyl sulfate radicals. The aliphatic groups may contain, in addition to carbon and hydrogen atoms, ether linkages as well as amido groups.

Suitable quaternary ammonium compounds may be mono-long chain alkyl, di-long chain alkyl, tri-long chain alkyl, and the like. Examples of such quaternary ammonium salts include behenalkonium chloride, behentrimonium chloride, behentrimonium methosulfate, benzalkonium chloride, benzethonium chloride, benzyl triethyl ammonium chloride, cetalkonium chloride, cetrimonium chloride, cetrimonium bromide, cetrimonium methosulfate, cetrimonium tosylate, cetylpyridinium chloride, dibehenyl/diarachidyl dimonium chloride, dibehenyldimonium chloride, dibehenyldimonium methosulfate, dicapryl/dicaprylyl dimonium chloride, dicetyldimonium chloride, and mixtures thereof.

Other quaternary ammonium salts useful as the cationic surfactant are compounds of the general formula:

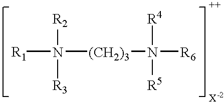

wherein R₁ is an aliphatic group having 16 to 22 carbon atoms, R₂, R₃, R₄, R₅, and R₆ are the same or different and are selected from H and alkyls having 1 to 4 carbon atoms and X is an anion as above defined.

Also, quaternary imidazolinium salts having the following general formula are also suitable:

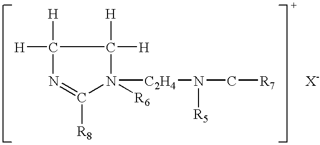

wherein R₅ is hydrogen or a C₁₋₄ alkyl; R₆ is a C₁₋₄ alkyl; R₇ is a C₈₋₂₂ alkyl; and R₈ is hydrogen, or a C₁₋₂₂ alkyl; and X is an anion as defined above.

Also suitable as the cationic conditioning agent are salts of fatty primary, secondary, or tertiary amines, wherein the substituted groups have 12 to 22 carbon atoms. Examples of such amines include dimethyl stearamine, dimethyl soyamine, stearylamine, myristylamine, tridecylamine, ethyl stearamine, and so on.

Also suitable as the cationic conditioning agent are cationic polymers such as:

Quaternary derivatives of cellulose ethers such as polymers sold under the tradename JR-125, JR-400, JR-30M. Preferred is Polyquaternium 10, which is a polymeric quaternary ammonium salt of hydroxyethyl cellulose reacted with a trimethyl ammonium subsituted epoxide.

Copolymers of vinylpyrrolidone having monomer units of the formula:

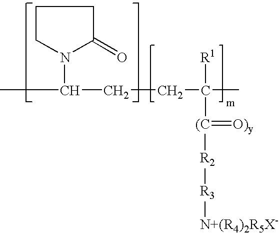

wherein R¹ is hydrogen or methyl, preferably methyl;

y is 0 or 1, preferably 1

R² is O or NH, preferably NH;

R³ is CₓH₂ₓ where x is 2 to 18, or —CH₂—CHOH—CH₂—, preferably CₓH₂ₓ where x is 2;

R⁴ is methyl, ethyl, phenyl, or C₁₋₄ substituted phenyl, preferably methyl; and

R⁵ is methyl or ethyl, preferably methyl.

Homopolymers of dimethyldiallylammonium chloride, or copolymer of dimethyldiallylammonium chloride and acrylamide. Such compounds are sold under the tradename MERQUAT™ by Merck and Company.

Homopolymers or copolymers derived from acrylic or methacrylic acid wherein the monomer units are selected from the group consisting of acrylamide, methylacrylamide, diacetone-acrylamide, acrylamide or methacrylamide substituted on the nitrogen by lower alkyl, alkyl esters of acrylic acid and methacrylic acid, vinylpyrrolidone, and vinyl esters.

Additional examples of cationic polymers that can be used in the compositions of the invention are the cationic polymers disclosed in U.S. Pat. Nos. 5,240,450 and 5,573,709, which are hereby incorporated by reference.

(ii) Other Ingredients

Other ingredients may be incorporated into the compositions including oily or waxy conditioning agents such as fatty acids, fatty alcohols, silicones, waxes, and the like.

(aa) Fatty Alcohols

If desired, the composition may contain about 0.1-20%, preferably 0.5-10%, more preferably 1-8% of a fatty alcohol having the formula RCH₂OH wherein R is a straight or branched chain saturated or unsaturated alkyl having at least about 6 to 30 carbon atoms. Examples of fatty alcohols suitable for use include behenyl alcohol, C₉₋₁₅ alcohols, caprylic alcohol, cetearyl alcohol, cetyl alcohol, coconut alcohol, decyl alcohol, lauryl alcohol, cetyl alcohol, myristyl alcohol, oleyl alcohol, palm alcohol, stearyl alcohol, tallow alcohol, C20-40 alcohols, and the like.

(bb) Silicone Conditioning Agents

Another optional ingredient may comprise one or more silicone conditioning agents. If present, suggested ranges are about 0.001-20%, preferably about 0.01-15%, more preferably about 0.1-10%. Suitable silicones include water soluble or water insoluble silicones, which may be volatile or non-volatile.

Suitable volatile silicones include cyclic or linear silicones. Suitable volatile silicones include cyclic silicones, linear silicones, or mixtures thereof. Cyclic silicones (or cyclomethicones) are of the general formula:

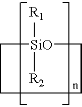

wherein n=3-7, and R₁ and R₂ are each independently H, C₁₋₈ alkyl, aryl, aralkyl, alkenyl, or a cylic or alicylic ring. Preferably R₁ and R₂ are each independently H or CH₃. Most preferably R₁ and R₂ are each CH₃.

Linear volatile silicones in accordance with the invention have the general formula:

(CH₃)₃—Si—O—[Si(CH₃)₂—O]ₙ—Si(CH₃)₃

where n=0-7, preferably 0-5.

Linear and cyclic volatile silicones are available from various commercial sources including Dow Corning Corporation and General Electric. The Dow Corning volatile silicones are sold under the tradenames Dow Corning 244, 245, 344, and 200 fluids. These fluids comprise octamethylcyclotetrasiloxane, decamethylcyclopentasiloxane, hexamethyldisiloxane, and mixtures thereof.

Suitable nonvolatile silicones include dimethicone, phenyl trimethicone, diphenyl dimethicone, hexadecyl methicone, cetyl dimethicone, and so on.

(cc) Other Ingredients

Also suitable for use in the conditioner are other ingredients such as humectants, biological agents, preservatives, antioxidants, sunscreens, and the like.

(c) Hair Color

The composition of the invention may be a hair color, specifically a permanent, semi-permanent, or temporary hair color. Typically such hair colors are in aqueous media and include a colorant in water, preferably with one or more surfactants and thickeners.

(i) Hair Colorant

If the composition is in the form of a hair colorant, the composition will comprise one more more dyes which are capable of coloring hair. If the hair color is permanent, the dye present will comprise at least one primary intermediate and, preferably, at least one coupler for the formation of oxidation dyes. Such primary intermediates and couplers are set forth in U.S. Pat. No. 5,843,193, which is hereby incorporated by reference in its entirety.

In the event the hair color is in the semi-permanent form, it will contain dyes such as direct dyes, basic dyes, and those known for use in semi-permanent hair color compositions.

In the event the hair color is in the temporary form, it will contain temporary dyes used in hair rinses which are well known in the art.

2. Compositions Applied to Skin

The composition used in the method of the invention may be in a form that is applied to skin, such as a cream, lotion, toner, astringent, foundation makeup, concealer, eyeshadow, blush, lipstick, lip gloss, lip conditioner, or the like. Such compositions may be in the aqueous form (e.g. in the form of a water-in-oil or oil-in-water emulsion, or in the anhydrous form).

(a) Lotions and Creams

The compositions may be in the form of lotions or creams that are applied to skin to provide moisturizing and conditioning properties. In such case, it is preferred that the creams and lotions be in the emulsion form. If in the water-in-oil or oil-in-water emulsion form the composition will comprise about 0.01-99.9%, preferably about 0.05-95%, more preferably about 1-80% water and 0.01-99.9%, about 0.05-95%, preferably 1-80% oil. Suitable skin creams and lotions may additionally contain the following ingredients, if desired.

(i) Day Time Protective Agent

The term “day time protective agent” means an agent that enhances the efficacy of the cream or lotion when used by a consumer that is engaging in typical day time activities. The day time protective agent may also enhance the efficacy of the composition in the amelioration of the effects of sun, wind and rain on the skin, and provide a synergistic effect with the silicone copolymer in improving moisture barrier properties of the skin or in ameliorating lines, wrinkles, skin imperfections, laxity, skin tone, age spots, skin discoloration, and the other negative effects associated with skin, including but not limited to those associated with estrogen loss (for example as found in menopause and peri-menopause).

The day time protective agent may include a chemical or physical sunscreen. Suitable day time protective agents include UVA and UVB chemical sunscreens and/or physical sunscreens.

(aa). UVA Chemical Sunscreens

The day time protective agent preferably comprises at least one UVA sunscreen. The term “UVA sunscreen” means a chemical compound that blocks UV radiation in the wavelength range of about 320 to 400 nm. Preferred UVA sunscreens are dibenzoylmethane compounds having the general formula:

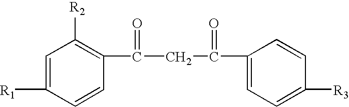

wherein R₁ is H, OR and NRR wherein each R is independently H, C₁₋₂₀ straight or branched chain alkyl; R₂ is H or OH; and R₃ is H, C₁₋₂₀ straight or branched chain alkyl.

Preferred is where R₁ is OR where R is a C₁₋₂₀ straight or branched alkyl, preferably methyl; R₂ is H; and R₃ is a C₁₋₂₀ straight or branched chain alkyl, more preferably, butyl.

Examples of suitable UVA sunscreen compounds of this general formula include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′diisopropylbenzoylmethane, 4-tert-butyl-4′-methoxydibenzoylmethane, 4,4′-diisopropylbenzoylmethane, 2-methyl-5-isorpoyl-4′-methoxydibenzoymethane, 2-metyl-5-tert-butyl-4′-methoxydibenzoylmethane, and so on. Particularly preferred is 4-tert-butyl-4′-methoxydibenzoylmethane, also referred to as Avobenzone. Avobenzone is commercial available from Givaudan-Roure under the trademark Parsol 1789, and Merck & Co. under the tradename Eusolex 9020.

The claimed compositions may contain from about 0.001-20%, preferably 0.005-5%, more preferably about 0.005-3% by weight of the composition of UVA sunscreen. Preferably, the UVA sunscreen is Avobenzone, and it is present at not greater than about 3% by weight of the total composition.

(bb). UVB Chemical Sunscreens

The term “UVB sunscreen” means a compound that blocks UV radiation in the wavelength range of from about 290 to 320 nm. A variety of UVB chemical sunscreens exist including α-cyano-β,β-diphenyl acrylic acid esters as set forth in U.S. Pat. No. 3,215,724, which is hereby incorporated by reference in its entirety. One particular example of a α-cyano-β,β-diphenyl acrylic acid ester is Octocrylene, which is 2-ethylhexyl 2-cyano-3,3-diphenylacrylate. In certain cases the composition may contain no more than about 10% by weight of the total composition of octocrylene. Suitable amounts range from about 0.001-10% by weight. Octocrylene may be purchased from BASF under the tradename Uvinul N-539.

Other suitable sunscreens include benzylidene camphor derivatives as set forth in U.S. Pat. No. 3,781,417, which is hereby incorporated by reference in its entirety. Such benzylidene camphor derivatives have the general formula:

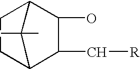

wherein R is p-tolyl or styryl, preferably styryl. Particularly preferred is 4-methylbenzylidene camphor, which is a lipid soluble UVB sunscreen compound sold under the tradename Eusolex 6300 by Merck.

Also suitable are cinnamate derivatives having the general formula:

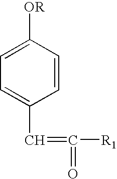

wherein R and R₁ are each independently a C! ₂₀ straight or branched chain alkyl. Preferred is where R is methyl and R₁ is a branched chain C₁₋₁₀, preferably C₈ alkyl. The preferred compound is ethylhexyl methoxycinnamate, also referred to as Octoxinate or octyl methoxycinnamate. The compound may be purchased from Givaudan Corporation under the tradename Parsol MCX, or BASF under the tradename Uvinul MC 80. Also suitable are mono-, di-, and triethanolamine derivatives of such methoxy cinnamates including diethanolamine methoxycinnamate. Cinoxate, the aromatic ether derivative of the above compound is also acceptable. If present, the Cinoxate should be found at no more than about 3% by weight of the total composition.

Also suitable as the UVB screening agents are various benzophenone derivatives having the general formula:

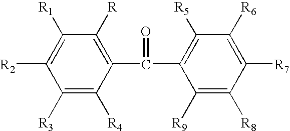

R through R₉ are each independently H, OH, NaO₃S, SO₃H, SO₃Na, Cl, R″, OR″ where R″ is C₁₋₂₀ straight or branched chain alkyl. Examples of such compounds include Benzophenone 1, 2, 3, 4, 5, 6, 7, 8, 9, 10, 11, and 12. Particularly preferred is where the benzophenone derivative is Benzophenone 3 (also referred to as Oxybenzone) and Benzophenone 4 (also referred to as Sulisobenzone), Benzophenone 5 (Sulisobenzone Sodium), and the like. Most preferred is Benzophenone 3.

Also suitable are certain menthyl salicylate derivatives having the general formula:

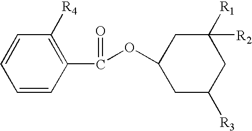

wherein R₁, R₂, R₃, and R₄ are each independently H, OH, NH₂, or C₁₋₂₀ straight or branched chain alkyl. Particularly preferred is where R₁, R₂, and R₃ are methyl and R₄ is hydroxyl or NH₂, the compound having the name homomenthyl salicylate (also known as Homosalate) or menthyl anthranilate. Homosalate is available commercially from Merck under the tradename Eusolex HMS and menthyl anthranilate is commercially available from Haarmann & Reimer under the tradename Heliopan. If present, the Homosalate should be found at no more than about 15% by weight of the total composition.

Various amino benzoic acid derivatives are suitable UVB absorbers including those having the general formula:

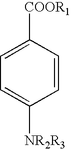

Wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂ and R₃ are H, or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula:

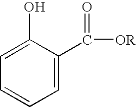

wherein R is a straight or branched chain alkyl, including derivatives of the above compound formed from mono-, di-, or triethanolamines. Particular preferred are octyl salicylate, TEA-salicylcate, DEA-salicylate, and mixtures thereof.

Generally, the amount of the UVB chemical sunscreen present may range from about 0.001-45%, preferably 0.005-40%, more preferably about 0.01-35% by weight of the total composition.

(cc). Physical Sunscreens

The day time protective agent may also include one or more physical sunscreens. The term “physical sunscreen” means a material that is generally particulate in form that is able to block UV rays by forming an actual physical block on the skin. Examples of particulates that serve as solid physical sunblocks include titanium dioxide, zinc oxide and the like in particle sizes ranging from about, 0.001-150 microns.

(i). The Night Time Protective Agent

The creams or lotions may contain one or more night time protective agents that enhance the beneficial properties of the silicone copolymer. The night time protective agent may synergistically act with the silicone copolymer to provide improved moisture barrier properties, in addition to the other benefits noted with respect to the silicone copolymer. Such ingredients may also be referred to as skin penetration enhancers in that they promote penetration of actives into the skin due to the substantive effect they provide on the skin. Examples of such materials include silicone oils, semi-solid or solid waxy materials, triglycerides, esters, paraffinic hydrocarbons, and the like. Preferably the night time protective agent is found in the claimed composition in ranges of from about 0.1-75%, preferably about 0.5-50%, more preferably about 1-40% by weight of the total composition. Such night time protective agents include those further described herein.

(aa). Silicone Oils

Particularly suitable as the protective agent are various types of silicone oils including water soluble or water insoluble volatile or non-volatile silicone oils as described with respect to the hair shampoo and conditioner, above.

(bb). Esters

Suitable protective agents also include various types of esters. In general such esters have the formula RCO—OR wherein each R is independently a C₁₋₂₅ straight or branched chain saturated or unsaturated alkyl, alkylcarbonyloxyalkyl, or alkoxycarbonylalkyl, aryl, which may be substituted or unsubstituted with halogen, hydroxyl, alkyl, and the like.

Examples of suitable esters include alkyl acetates, alkyl behenates, alkyl lactates, alkyl benzoates, alkyl octanoates, alkyl salicylates, and in particular C₁₂₋₁₅ alkyl benzoate. Examples of further esters are set forth on pages 1670-1676 of the CTFA International Cosmetic Ingredient Handbook, Eighth Edition, 2000, which is hereby incorporated by reference.

(cc). Fats and Oils

Fats and oils are also suitable as protective agents. They may be further defined as glyceryl esters of fatty acids (triglycerides), as well as the synthetically prepared esters of glycerin and fatty acids having the following general formula:

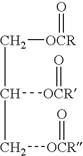

wherein R, R′, and R″ are each independently fatty acid radicals. Examples of such materials include oils such as apricot kernel oil, avocado oil, canola oil, olive oil, sesame oil, peanut oil, trilinolenin, trilinolein, trioctanoin, tristearin, triolein, sesame oil, rapeseed oil, sunflower seed oil, and so on.

(dd). Fatty Acids

Fatty acids may also serve as protective agents. Fatty acids are the carboxylic acids obtained by hydrolysis of animal or vegetable fats and oils. Carboxylic acids having alkyl chains shorter than about seven carbon atoms are not generally considered fatty acids. Fatty acids have the general structure R—COOH where R is a straight or branched chain saturated or unsaturated C₇₋₆₅ alkyl. Examples of suitable fatty acids include arachidic acid, arachidonic acid, behenic acid, capric acid, caproic acid, caprylic acid, coconut acid, corn acid, cottonseed acid, hydrogenated coconut acid, hydroxystearic acid, lauric acid, linoleic acid, linolenic acid, linseed acid, myristic acid, oleic acid, palmitic acid, palm kernel acid, soy acid, tallow acid, and the like.

(ee). Fatty Alcohols

Fatty alcohols may also be suitable as the night time protective agent, as set forth with respect to the hair conditioner above.

(ff). Hydrocarbons

Hydrocarbons also serve as good skin penetrants for use as the night time protective agent. Hydrocarbons are generally chemically inert. Examples of suitable hydrocarbons include C₇₋₆₀ isoparaffins, ethane, heptane, hexane, hydrogenated polyisobutene, isobutane, isododecane, isoeicosane, isohexadecane, isopentane, microcrystalline wax, mineral oil, mineral spirits, paraffin, petrolatum, petroleum distillates, squalene, polyethylene, and mixtures thereof. Preferred hydrocarbons are mineral oil and polyethylene.

(iii). Other Ingredients

The creams and lotions may contain other ingredients including botanical extracts, humectants, preservatives, polymers, particulates, surfactants, and the like.

(aa). Organic Surfactants

Suitable organic surfactants include those having anionic, nonionic, amphoteric, zwitterionic, or cationic moieties and are as set forth with respect to the hair conditioner and hair shampoo, above. The organic surfactant, if present, should range from about 0.001-30%, preferably about 0.005-25%, preferably about 0.01-20% by weight of the total composition. Particularly preferred are one or more nonionic surfactants or emulsifiers including alkoxylated alcohols, or ethers, formed by the reaction of an alcohol with an alkylene oxide, usually ethylene or propylene oxide. Preferably the alcohol is either a fatty alcohol having 6 to 30 carbon atoms.

Suitable cationic, anionic, zwitterionic, and amphoteric surfactants are disclosed in U.S. Pat. No. 5,534,265, which is hereby incorporated by reference in its entirety.

(bb). Humectants

The skin creams and lotions may comprise 0.01-30%, preferably 0.5-25%, more preferably 1-20% by weight of the total composition of one or more humectants. Suitable humectants include materials such as glycols, sugars, and the like. Suitable glycols include polyethylene and polypropylene glycols such as PEG 4-240, which are polyethylene glycols having from 4 to 240 repeating ethylene oxide units; as well as C₁₋₆ alkylene glycols such as propylene glycol, butylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Preferably, the humectants used in the composition of the invention are C₁₋₆, preferably C₂₋₄ alkylene glycols, most particularly butylene glycol.

(cc). Other Botanical Extracts

It may be desirable to include one or more additional botanical extracts in the compositions. If so, suggested ranges are from about 0.0001 to 10%, preferably about 0.0005 to 8%, more preferably about 0.001 to 5% by weight of the total composition. Suitable botanical extracts include extracts from plants (herbs, roots, flowers, fruits, seeds) such as flowers, fruits, vegetables, and so on, including acacia (dealbata, famesiana, senegal), acer saccharinum (sugar maple), acidopholus, acorus, aesculus, agaricus, agave, agrimonia, algae, aloe, citrus, brassica, cinnamon, orange, apple, blueberry, cranberry, peach, pear, yam, soy, lemon, lime, pea, seaweed, green tea, chamomile, willowbark, mulberry, poppy, and those set forth on pages 1646 through 1660 of the CTFA Cosmetic Ingredient Handbook, Eighth Edition, Volume 2.

(dd). Gellants

It may be desireable to include other gellants in the oil or water phase of the composition to provide gelling or thickening of the composition. Such gellants may be included a range of about 0.1-20%, preferably about 1-18%, more preferably about 2-10% by weight of the total composition is suggested. Suitable gellants include soaps, i.e. salts of water insoluble fatty acids with various bases. Examples of soaps include the aluminum, calcium, magnesium, potassium, sodium, or zinc salts of C₆₋₃₀, preferably C₁₀₋₂₂ fatty acids.

Also suitable are hydrocolloids such as gellan gum, gum arabic, carrageenan, and those set forth in U.S. Pat. No. 6,197,319 which is hereby incorporated by reference in its entirety.

Water soluble synthetic polymeric materials are also good gellants, including polymers of acrylic acid or C₁₋₂₀ esters thereof, which may be crosslinked or uncrosslinked. Examples include Carbopol (polymer of acrylic acid crosslinked with a polyfunctional agent which is the allyl ether of sucrose or the allyl ether of pentaerythritol), and the like.

Other ingredients are also suitable for inclusion in lotions and creams, such as preservatives, antioxidants, and the like.

(b) Lip Compositions

In one of the preferred embodiments of the invention, the cosmetic composition is in the form of a composition that is applied to the lips to provide color, gloss, or conditioning, such as a lipstick, lip gloss, or lip conditioner. Such lip compositions may be in the emulsion form, but are preferably anhydrous. Such lip compositions generally comprise a particulate material such as pigment or particulate fillers in a oil or wax base.

(i) Oils

Oils suitable for use in preparation of the lip composition include silicone oils, organic oils, or mixtures thereof. The silicone oils can be water insoluble or water soluble, or volatile or non-volatile. Suitable silicone oils are those set forth above with respect to the hair shampoo and conditioner and skin creams and lotions. Suitable organic oils include the esters, fats, oils, and hydrocarbons set forth above, and in the same ranges as are found in the skin creams and lotions.

In one preferred embodiment of the invention the lip composition is a transfer resistant lipstick in the solid stick, semi-solid, or liquid form, further comprising about:

0.1-80% of a volatile solvent,

1-25% of the silicone copolymer; and

0.001-35% pigment.

(ii) Pigments

The lip composition of the invention may contain 0.001-35%, preferably 0.01-20% more preferably 0.1-10%, by weight of the total composition pigments. The organic pigments are generally various aromatic types including azo, indigoid, triphenylmethane, anthraquinone, and xanthine dyes which are designated as D&C and FD&C blues, browns, greens, oranges, reds, yellows, etc. Organic pigments generally consist of insoluble metallic salts of certified color additives, referred to as the Lakes. Inorganic pigments include iron oxides, ultramarines, chromium, chromium hydroxide colors, and mixtures thereof.

(iii) Particulate Fillers

The lip composition may comprise one or more particulate fillers that may be colored or non-colored (for example white). If present, suggested ranges for particulate fillers are about 0.001-20%, preferably about 0.01-18%, more preferably about 0.1-15%.

Suitable particulate fillers include powders such as bismuth oxychloride, titanated mica, fumed silica, spherical silica, polymethylmethacrylate, micronized teflon, boron nitride, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The above mentioned powders may be surface treated with lecithin, amino acids, mineral oil, silicone oil or various other agents either alone or in combination, which coat the powder surface and render the particles more lipophilic in nature. Some of the powders may be in the form of fibers, which are particulates that have a cross-sectional area and at least some lengthwise dimension, possibly as small as 0.1 mm.

The composition may contain a mixture of both pigmented and non-pigmented powders. The percentage of pigments used in the powder component will depend on the type of cosmetic being formulated.

(iv) Structuring Agents

It may be desirable to incorporate one or more structuring agents in the lip composition, particularly if it is desired to formula a lip composition that is in the solid or semi-solid form. Suitable structuring agents provide viscosity or thickness to the lip composition and include waxes or certain other types of gelling agents. Suggested ranges of structuring agent are about 0.001-60%, preferably about 0.01-50%, more preferably about 0.1-45%.

(aa) Waxes

Examples of wax structuring agents include natural, synthetic, or silicone waxes. Natural waxes include animal, vegetable, or mineral waxes. Fatty alcohols and fatty acids as set forth above with respect to skin creams and lotions are suitable for use as the waxy structuring agent. Particularly preferred in the lip compositions are one or more fatty alcohol waxy structuring agents, such as C20-40 alcohols.

Also suitable are synthetic waxes, preferably ethylene homo- or copolymers. The molecular weight of the ethylene homopolymer and/or copolymers used as the wax component may vary, so long as the melting point of the homo- or copolymer either alone or in combination is not greater than 135° C. Generally polyethylene waxes having a melting point range of 30 to 135° C. will have a molecular weight ranging from about 100 and 2,000. Preferably the ethylene copolymers are comprised of ethylene monomer units in either repetitive or randon sequence, in combination with monomer units of the following formula:

CH₂═CH—R₁

wherein R₁ is a C₁₋₃₀ straight or branched chain saturated or unsaturated alkyl, aryl, or aralkyl, preferably a C₁₋₁₀ straight or branched chain alkyl. Examples of ethylene homo- and copolymers which may be used in the invention are set forth in U.S. Pat. No. 5,556,613, which is hereby incorporated by reference.

Hydrocarbon waxes are also suitable structuring agents, including petrolatum, microcrystalline wax, hydrogenated polyisobutene, paraffin, red petrolatum, squalene, squalane, and the like.

Other oleaginous materials that are solid or semi-solid at room temperature and have a melting point of 32 to 100° C. may be used. Examples of such materials include lanolin and derivatives thereof such as lanolin alcohol, acetylated lanolin alcohol; or hydrogenated oils, such as hydrogenated castor oil, or alkoxylated hydrogenated castor oil. Preferably, one of the oil phase gelling agents is PEG-40 hydrogenated castor oil.

Suitable structuring agents include animal or plant waxes such as apple wax, avocado wax, bayberry wax, carnauba wax, ceresin, beeswax, hydrogenated jojoba oil, hydrogenated jojoba wax, hydrogenated rice bran wax, hydrolyzed beeswax, jojoba butter, jojoba esters, jojoba wax, lanolin wax, mink wax, montan acid wax, montan wax, ouricury wax, ozokerite, palm kernal wax, PEG-5-20 beeswax, PEG-12 carnauba, Rice wax, shellac wax, spent grain wax, sulfurized jojoba oil, synthetic beeswax, synthetic candellila wax, synthetic carnauba, synthetic jojoba wax, and mixtures thereof.

(bb) Hydrophobically Modified Materials

Also suitable as structuring agents are hydrophobically modified materials such as hydrophobically modified silica, i.e. silica modified by substitution of a sufficient number of the hydroxyl groups with hydrophobic C₁₋₄ alkyl groups, preferably methyl.

(v) Additional Film Formers

In the most preferred embodiment of the lip composition, the composition additionally comprises one or more additional film forming polymers in addition to the silicone copolymer. These film forming polymers may include synthetic polymers such as silicones and polymers made from ethylenically unsaturated monomers. Also suitable are natural polymers such as gums, resins, and similar materials derived from natural sources.

(aa) Silicone Film Formers

Suitable silicone film formers include siloxane resins, silicone ester waxes, and the like. Preferably the lip composition comprises about 0.01-50%, preferably 0.5-40%, more preferably 1-35% by weight of the total composition of one or more polymeric film formers in addition to the silicone copolymer.

Suitable silicone film forming polymers include linear and cross-linked silicones that are resinous in character, e.g. which exhibit properties generally associated with resins such as film forming capability and substantivity. An example of one type of cross linked silicone is an MQ resin, which is a siloxy silicate polymer having the following general formula:

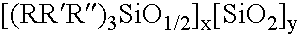

wherein R, R′ and R″ are each independently a C₁₋₁₀ straight or branched chain alkyl or phenyl, and x and y are such that the ratio of (RR′R″)₃SiO_{1/2} units to SiO₂ units ranges from about 0.5 to 1 to 1.5 to 1. Preferably R, R′ and R″ are a C₁₋₆ alkyl, and more preferably are methyl and x and y are such that the ratio of (CH₃)₃SiO_{1/2} units to SiO₂ units is about 0.75 to 1. Most preferred is this trimethylsiloxysilicate containing 2.4 to 2.9 weight percent hydroxyl groups which is formed by the reaction of the sodium salt of silicic acid, chlorotrimethylsilane, and isopropyl alcohol. The manufacture of trimethylsiloxysilicate is set forth in U.S. Pat. Nos. 2,676,182; 3,541,205; and 3,836,437, all of which are hereby incorporated by reference. Trimethylsiloxysilicate as described is available from GE Silicones under the tradename SR-1000, which is a solid particulate material. Also suitable is Dow Corning 749 which is a mixture of volatile cyclic silicone and trimethylsiloxysilicate.

Also suitable are T or MT resins, which have repeating T and, optionally M units.

Such resins have the general formula:

[R₁R₂R₃SiO_{1/2}]ₓ[R₁SiO_{3/2}]_{y}

wherein R₁, R₂, and R₃ are each independently as defined above, and x ranges from 0-10,000; and y ranges from 1-10,000. Examples of such resins include MK Resin made by Particularly preferred are polysilsesquioxanes manufactured by Wacker Chemie under the Resin MK designation. This polysilsesquioxane is a polymer comprise of T units and, optionally one or more D (preferably dimethylsiloxy) units. This particularly polymer may have ends capped with ethoxy groups, and/or hydroxyl groups, which may be due to how the polymers are made, e.g. condensation in aqueous or alcoholic media. Other suitable T resins include those manufactured by Shin-Etsu Silicones as the “KR” series, e.g. KR-220L, 242A, and so on. These particular silicone resins may contain endcap units that are hydroxyl or alkoxy groups which may be present due to the manner in which such resins are manufactured.

Also suitable are linear, high molecular weight silicones that are semi-solids, solids, or gums at room temperature. Examples of such silicones include dimethicones having viscosities ranging from 500,000 to 10 million or dimethicone copolyols.

Also suitable are silicone esters as disclosed in U.S. Pat. Nos. 4,725,658 and U.S. Pat. No. 5,334,737, which are hereby incorporated by reference. Such silicone esters comprise units of the general formula RₐR^{E}_{b}SiO_{[4-(a+b)/2}] or R¹³ₓR^{E}_{y}SiO_{1/2}, wherein R and R¹³ are each independently an organic radical such as alkyl, cycloalkyl, or aryl, or, for example, methyl, ethyl, propyl, hexyl, octyl, decyl, aryl, cyclohexyl, and the like, a is a number ranging from 0 to 3, b is a number ranging from 0 to 3, a+b is a number ranging from 1 to 3, x is a number from 0 to 3, y is a number from 0 to 3 and the sum of x+y is 3, and wherein R^{E} is a carboxylic ester containing radical. Preferred R_{E} radicals are those wherein the ester group is formed of one or more fatty acid moieities (e.g. of about 2, often about 3 to 10 carbon atoms) and one or more aliphatic alcohol moieities (e.g. of about 10 to 30 carbon atoms). Examples of such acid moieties include those derived from branched-chain fatty acids such as isostearic, or straight chain fatty acids such as behenic. Examples of suitable alcohol moieties include those derived from monohydric or polyhydric alcohols, e.g. normal alkanols such as n-propanol and branched-chain etheralkanols such as (3,3,3-trimethylolpropoxy)propane. Preferably the ester subgroup (i.e. the carbonyloxy radical) will be linked to the silicon atom by a divalent aliphatic chain that is at least 2 or 3 carbon atoms in length, e.g. an alkylene group or a divalent alkyl ether group. Most preferably that chain will be part of the alcohol moiety, not the acid moiety. Such silicones may be liquids or solids at room temperature.

(bb) Polymers From Ethylenically Unsaturated Monomers

Other synthetic polymeric film formers that may be used for the lip composition are polymers made from various types of ethylenically unsaturated monomers, including esters of acrylic or methacrylic acid, or ethylene homo- or copolymers, or styrene homo- or copolymers. Such polymers may be homopolymers, copolymers, or graft or block copolymers. Examples of such polymeric film formers are disclosed in U.S. Pat. No. 6,143,283 and 6,153,206 which are hereby incorporated by reference in their entirety.

U.S. Pat. No. 6,143,283 teaches acrylic polymers that are adhesives at room temperature. One type of such polymer comprises a backbone of vinyl, methacrylic, or acrylic monomers and pendant siloxane and fluorochemical groups, for example Poly(isobutyl methacrylate)-co-methyl FOSEA sold by 3M Company under the tradename SA 70-5 IBMMF. Also suitable are vinyl silicone graft or block copolymers, such as poly(dimethylsiloxane)-g-poly(isobutyl) methacrylate sold by 3M Company under the tradename VS 70.

Also suitable are the methacrylate copolymers set forth in U.S. Pat. No. 6,153,206, which comprise uncrosslinked synthetic polymers comprising a first repeat unit selected from methacrylate ester monomers which, if polymerized, would yield a polymer having a glass transition temperature of −10 to 75° C., and a second repeat unit selected from methacrylate ester monomers which, if polymerized, would yield a polymer having a glass transition temperature of 76 to 120° C. Examples of such copolymers include those where the first repeat unit is selected from isobutyl methacrylate, ethyl methacrylate, hexyl methacrylate, and mixtures thereof; and the second repeat unit is selected from methyl methacrylate, isobornyl methacrylate or mixtures thereof. These polymers from ethylenically unsaturated monomers may be co-polymerized with one or more organic groups. One type of organic group that can be polymerized with the above monomers includes a urethane monomer. Urethanes are generally formed by the reaction of polyhydroxy compounds with diisocyanates, as follows:

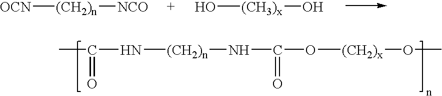

wherein x is 1-1000.

Another type of monomer that may be polymerized with the above comprise amide groups, preferably having the the following formula:

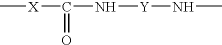

wherein X and Y are each independently linear or branched alkylene having 140 carbon atoms, which may be substituted with one or more amide, hydrogen, alkyl, aryl, or halogen substituents.

(cc) Natural Film Forming Polymers

Various types of natural film forming polymers may be used as well. Examples of such polymers include cellulosics and derivatives thereof, gums such as gum arabic, and resins obtained from trees and plants.

Preferably, the lip compositions comprise an additional film forming polymer which is a silicone, more particularly a silicone resin which is trimethylsiloxysilicate or polymethylsilsesquioxane.

(c) Foundation Makeup

The carrier composition may comprise a foundation makeup composition, which may be in the anhydrous or emulsion form. If the emulsion form, the composition may be a water-in-oil or oil-in-water emulsion comprising about 0.1-95%, preferably about 0.5-85%, more preferably about 1-75% water and about 0.5-35%, preferably about 1-25%, more preferably about 1.5-20% oil. The ingredients that may be found in this composition are further described below.

The oils present in the composition may be volatile, non-volatile or mixtures thereof. Preferably the total oil content is in the range of about 0.5-75%, preferably 1-70%, more preferably 5-65% by weight of the total composition. Suitable oils include those set forth under the hair conditioner, hair shampoo, creams and lotions, and lip compositions.

The foundation may also comprise one or more pigments or particulate fillers or both. If present, such pigments and particulate fillers are as set forth above with respect to the skin care and lip compositions, and in the same percentage ranges.

The other ingredients mentioned for use in the skin creams and lotions, shampoo, conditioner, and lip compositions are also suitable for use in the foundation makeup and in the same percentage ranges.

(d) Color Cosmetics

The cosmetically acceptable carrier also includes color cosmetic products such as blush, eyeshadow, concealer, and mascara. These products may be in the anhydrous or emulsion form, and may contain any of the ingredients in the percentage ranges as set forth for the other cosmetics mentioned herein.

(e) Nail Products

The cosmetically acceptable carrier for use in the method of the invention may also include various types of nail products such as cuticle creams, lotions, or conditioners, nail enamel (both colored and clear).

II. The Methods

The claimed compositions may be used in a method to improve the moisture barrier properties of keratinous surfaces, improve the appearance of skin imperfections, lines, wrinkles, sags, age spots, and discolorations, and to improve the aesthetic properties such as gloss, shine, wear, and spreadability of cosmetic compositions.

A. A Method for Improving Moisture Barrier Properties.

Most notably, use of the silicone copolymer in cosmetic compositions will improve the moisture barrier properties of the keratinous surface to which the cosmetic composition is applied. Retention of moisture within keratinous surfaces such as the skin, nails, lips, hair, etc. is important to maintaining hydration and health, and resisting dryness of the surface. Moisture retention in the skin is particularly desirable when using cosmetic compositions on the facial or body skin or lips. Particularly with respect to lipsticks, it is very desirable to retain moisture in the lips to prevent dryness and chapping. Transfer resistant lipsticks in particular may tend to be more drying to the lips and use of the silicone copolymer in compositions applied to the lips causes improvement in the moisture barrier properties because the film formed by the copolymer will cause the lips to better retain internal moisture and hydration.

It is not necessary that the film formed by the composition be completely occlusive. Partial occlusiveness is sufficient to improve moisture barrier properties and reduce trans epithelial water loss in the keratinous surface to which the composition containing the silicone copolymer is applied.

The composition should be applied to the desired keratinous surface at least once per day, or as many times a day as desired, depending on the type of composition that is being applied. For example, if the composition is a lip composition, it may be applied to the lips many times a day. On the other hand if the composition is a hair conditioner or shampoo, once a day or once every other day is normal.

B. Method For Improving Appearance of Skin

In addition, the silicone copolymer, when used in a cosmetic composition that is applied to a keratinous surface, will physically improve the appearance of the surface in a variety of ways such as minimizing the appearance of skin imperfections, and reducing the appearance of lines, wrinkles, sags, discoloration, or dryness. The silicone copolymer in the cosmetically acceptable carrier is applied to the skin in the desired form. The frequency and amount of application will depend on the cosmetic vehicle that is used. Obviously, if the composition is in the form of a skin cream or lotion, it will be applied to the skin once or twice a day. On the other hand, if the cosmetic composition is in the form of a lipstick, it may be applied to the skin many times a day.

C. Method for Improving Aesthetics of Cosmetic Films

The claimed method will also improve the aesthetics of cosmetic films that are applied to lips. The term “improve aesthetics” means properties that have an impact on the physical appearance and feel of the film on the lips. Aesthetics that are improved with the silicone copolymer used in the invention include gloss, shine, transfer resistance, and spreadibility. Cosmetic lip compositions that contain the silicone copolymer will exhibit improved aesthetics such as gloss, transfer resistance, shine, and spreadibility.

The invention will be further described in connection with the following examples which are set forth for the purposes of illustration only.

### EXAMPLE 1

A transfer resistant lipstick composition was made according to the following formula:

| | w/w % |
|---|---|
| C20-40 alcohol wax | 14.60 |
| Silicone polymer* | 16.20 |
| Trimethylsiloxysilicate | 8.10 |
| Isododecane | 51.10 |
| Pigments | 10.00 |

*Dow Corning 7-4505, a siloxane resin/diorganosiloxane copolymer identified by CAS number 68440-70-0 - trimethylated silica treated with dimethyl siloxane.

The composition was prepared by heading the wax to 90° C. and adding the remaining ingredients and mixing well. The composition was poured into a stick mold and allowed to cool. The stick was applied to the lips and exhibited good transfer resistance. When covered with a gloss coat of a thin layer of hydrocarbon oil the composition remained on the lips. Two panelists wore the lipstick. One panelist reported that most of it was removed after eating a meal. The second panelist reported that the film pieced after three hours.

### EXAMPLE 2

A lash color was made according to the following formula:

| | w/w % |
|---|---|
| Isododecane | 8.18 |
| Dimethicone (1 centipoise) | 8.18 |
| Polysilicone-6 | 20.35 |
| Dimethicone/dimethicone crosspolymer | 6.00 |
| Cyclomethicone/dimethiconol/trihydroxystearin (86:14) | 10.00 |
| Dow Corning 7-4400 PSA in isododecane (50:50) | 10.00 |
| Dibutyl adipate | 2.4 |
| Methyl paraben | 0.3 |
| Propyl paraben | 0.1 |
| Dehydroacetic acid | 0.2 |
| Sorbic acid | 0.03 |
| Silica | 7.20 |
| Silica shell | 1.2 |
| FD&C Red #40 aluminum lake | 1.74 |
| FD&C Blue #1 aluminum lake | 1.74 |
| FD&C Yellow #5 aluminum lake | 0.73 |
| D&C Green #5 | 0.05 |
| Quaternium-18 hectorite/isododecane/ | 21.6 |
| propylene glycol | |

The composition was prepared by combining the ingredients and mixing well. The composition was poured into a vial.

### EXAMPLE 3

A foundation makeup was made according to the following formula:

| | w/w % |
|---|---|
| Cyclopentasiloxane/dimethicone copolyol | 19.50 |
| Dow Corning 7-4505 silicone adhesive* | 10.70 |
| Cyclomethicone | 6.00 |
| Sorbitan sesquioleate | 0.05 |
| Propyl paraben | 0.10 |
| Iron oxides/titanium dioxide/methicone | 8.00 |
| Red iron oxide/boron nitride | 0.98 |
| Yellow iron oxide/methicone | 1.13 |
| Black iron oxide/methicone | 0.17 |
| Mica/methicone | 1.21 |
| Silk powder | 0.10 |
| Boron nitride | 3.51 |
| Nylon-12 | 2.0 |
| Bisabolol | 0.05 |
| Tribehenin | 1.5 |
| Dimethicone | 1.0 |
| Cetyl dimethicone copolyol | 0.25 |
| Solution of glyceryl rosinateC9-11 Isoparaffin (50/50) | 2.5 |
| Isododecane | 1.0 |
| Polyisobutene | 1.0 |
| Water | QS 100 |
| Butylene glycol | 4.5 |
| Methyl paraben | 0.2 |
| Tetrasodium EDTA | 0.01 |
| Sodium chloride | 1.0 |
| Ethanol | 3.0 |

*70 parts adhesive and 30 parts isododecane

The composition was prepared by combining the ingredients and mixing well to emulsify.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A cosmetic composition for application to keratinous surfaces for the purpose of coloring, conditioning, or protecting such keratinous surface, comprising a silicone polymer which is the reaction product of a siloxane resin and an diorganosiloxane, in a cosmetically acceptable carrier.

2. The composition of claim 1 wherein the siloxane resin is a MQ resin, a T resin, or an MT resin.

3. The composition of claim 2 wherein the siloxane resin is an MQ resin.

4. The composition of claim 1 wherein the diorganosiloxane is polydimethylsiloxane.

5. The composition of claim 4 wherein the diorganosiloxane has hydroxy functional terminal groups.

6. The composition of claim 5 wherein the silicone resin has hydroxy functional terminal groups.

7. The composition of claim 6 wherein the hydroxy functional terminal groups of the siloxane resin and the diorganosiloxane react in the presence of ammonia and heat to form the silicone polymer.

8. The composition of claim 1 which is in the form of an emulsion.

9. The composition of claim 8 which is a lotion or cream.

10. The composition of claim 8 which is a foundation makeup.

11. The composition of claim 1 which is an anhydrous pigmented composition.

12. The composition of claim 11 wherein the anhydrous pigmented composition is a lipstick.

13. The composition of claim 12 wherein the lipstick comprises a volatile solvent, pigments, and the polymer.

14. The composition of claim 1 which is a mascara.

15. The composition of claim 14 which is in the emulsion form.

16. A method for improving aesthetics such as gloss, transfer resistance, spreading, and wear of cosmetic compositions applied to lips comprising including in said cosmetic composition at least one silicone polymer which is the reaction product of a siloxane resin and a diorganosiloxane.

17. The method of claim 16 wherein the siloxane resin has hydroxy functional terminal groups.

18. The method of claim 17 wherein the diorganosiloxane has hydroxy functional terminal groups.

19. The method of claim 18 wherein the hydroxy terminal functional groups of the siloxane resin and the diorganosiloxane react in the presence of ammonia and heat to form the polymer.

20. A transfer resistant lipstick composition comprising, a silicone copolymer which is the reaction product of a diorganosiloxane and a silicone resin, a volatile oil, and pigments.
